(19) United States
(12) Patent Application Publication
(10) Pub. No.: US 2019/0091189 A1
(43) Pub. Date: Mar. 28, 2019

(54) ADMINISTRATION OF EXOGENOUS KETONE TO LOWER BLOOD GLUCOSE

(71) Applicants: Csilla Ari D'Agostino, Tampa, FL (US); Dominic Paul D'Agostino, Tampa, FL (US)

(72) Inventors: Csilla Ari D'Agostino, Tampa, FL (US); Dominic Paul D'Agostino, Tampa, FL (US)

(21) Appl. No.: 16/086,748

(22) PCT Filed: Mar. 21, 2017
(86) PCT No.: PCT/US2017/023450

§ 371 Date: Sep. 20, 2018

Related U.S. Application Data
(60) Provisional application No. 62/311,108, filed on Mar. 21, 2016.
(60) Provisional application No. 62/317,057, filed on Apr. 1, 2016.

(51) Int. Cl. A61K 31/225; A61K 31/23; A61K 31/19; A61K 9/00; A61P 3/10

(52) U.S. Cl. CPC A61K 31/225; A61K 31/23; A61P 3/10; A61K 9/0053; A61K 31/19

(57) ABSTRACT

Provided herein are compositions and methods for reducing blood glucose in a subject.

### CROSS-REFERENCE TO RELATED APPLICATIONS

This application claims priority to U.S. Provisional Application No. 62/311,108, filed on Mar. 21, 2016 and to U.S. Provisional Application No. 62/317,057 filed on Apr. 1, 2016, which are incorporated fully herein by reference.

### TECHNICAL FIELD

The present disclosure relates to compositions for lowering blood glucose levels in a subject.

### BACKGROUND

The primary fuel source for the human body is glucose, a sugar that is metabolized in the liver to yield acetyl-CoA, which drives the citric acid cycle to produce ATP. In the absence of glucose, the body switches to metabolizing fatty acids for fuel, generating ketone bodies that can be transported out of the liver to other tissues in the body. The ketone bodies are then converted back to acetyl-CoA in the mitochondria of the glucose-deprived tissue, allowing the citric acid cycle to continue generating ATP when glucose supplies are low.

The ketogenic diet (KD) consists of a macronutrient ratio that induces a metabolic shift towards fatty acid oxidation and hepatic ketogenesis, elevating the ketone bodies acetoacetate (AcAc), β-hydroxybutyrate (βHB), and acetone in the blood. These ketone bodies serve as alternative metabolic substrates during the prolonged reduction of glucose that occurs during the ketogenic diet.

Emerging evidence supports the therapeutic potential of the ketogenic diet (KD) for a variety of disease states, including epilepsy, diabetes, acne, polycystic ovary syndrome (PCOS), cancer, amyotrophic lateral sclerosis (ALS), traumatic brain injury (TBI) and Alzheimer's disease (AD). However, several factors limit the efficacy and utility of the ketogenic diet as metabolic therapy for widespread clinical use. Patient compliance to the KD can be low due to the severe dietary restriction or intolerance to high-fat ingestion. Maintaining ketosis can be difficult as consumption of even a small quantity of carbohydrates or excess protein can rapidly inhibit ketogenesis. Enhanced ketone body production and tissue utilization can take several weeks and patients may experience hypoglycemic symptoms during this transitional period. In addition, concerns have been raised about ketogenic diets increasing total cholesterol and triglycerides while decreasing high density lipoprotein (HDL) levels, leading to diminished cardiovascular health. As such, alternative methods to establish and maintain ketosis in a patient without the negative side effects of the ketogenic diet are needed.

### SUMMARY

The present disclosure describes a method to reduce blood glucose in a subject, comprising administering a composition comprising one or more ketogenic compounds to the subject. The one or more ketogenic compounds may be selected from the group consisting of a ketone ester, a ketone salt, a ketone body precursor, and a combination thereof. The ketone ester may be 1,3-butanediol-acetoacetate diester. The ketone salt may be Na⁺/K⁺ βHB mineral salt. The composition may reduce blood glucose in the subject with or without exercise. The subject may be a human.

### BRIEF DESCRIPTION OF THE DRAWINGS

FIGS. 1A-C shows blood BHB levels in rats following administration of ketogenic compounds. FIG. 1A shows blood BHB levels in Sprague-Dawley rats following chronic administration of ketogenic compounds. FIG. 1B shows blood BHB levels in Sprague-Dawley rats 24 hours and 7 days after administration of ketogenic compounds. FIG. 1C shows blood BHB levels in WAG/Rij rats 24 hours and 7 days after administration of ketogenic compounds.

FIGS. 2A-C shows blood glucose levels in rats following administration of ketogenic compounds. FIG. 2A shows blood glucose levels in Sprague-Dawley rats following chronic administration of ketogenic compounds. FIG. 2B shows blood glucose levels in Sprague-Dawley rats 24 hours and 7 days after administration of ketogenic compounds. FIG. 2C shows blood glucose levels in WAG/Rij rats 24 hours after administration of ketogenic compounds.

FIG. 3 shows blood BHB levels in exercised Sprague-Dawley rats 24 hours and 7 days after administration of ketogenic compounds.

FIG. 4 shows blood glucose levels in exercised Sprague-Dawley rats 24 hours and 7 days after administration of ketogenic compounds.

FIG. 5 shows blood BHB levels in exercised Sprague-Dawley rats 30-45 minutes after administration of ketogenic compounds.

FIG. 6 shows blood glucose levels in exercised Sprague-Dawley rats 30-45 minutes after administration of ketogenic compounds.

FIGS. 7A and 7B show blood levels of glucose in WAG/Rij rats 60 minutes after administration of ketogenic compounds.

FIGS. 8A and 8B show blood BHB levels in WAG/Rij rats 60 minutes after administration of ketogenic compounds.

FIGS. 9A and 9B show the blood glucose levels in exercised WAG/Rij rats after the first and seventh administration of ketogenic compounds.

FIGS. 10A and 10B show the blood BHB levels in exercised WAG/Rij rats after the first and seventh administration of ketogenic compounds.

FIGS. 11A-D shows the effects of ketogenic compounds on total cholesterol (FIG. 11A), Triglycerides (FIG. 11B), LDL (FIG. 11C), and HDL (FIG. 11D).

FIG. 12 shows blood BHB levels in G1D Mice after administration of ketogenic compounds.

FIG. 13 shows blood glucose levels in G1D Mice after administration of ketogenic compounds.

### DETAILED DESCRIPTION

Certain disease conditions, including inflammation and oxidative stress, are accelerated by high blood glucose levels. Powerful homeostatic mechanisms regulate blood glucose levels, such that lowering blood glucose is difficult. Additionally, blood glucose is generally elevated after exercise. The present disclosure describes a method to reduce blood glucose in a subject, comprising administering to the subject a composition comprising one or more ketogenic compounds. The disclosed method may be used to reduce blood glucose with or without exercise.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The term “administration” or “administering” is used throughout the specification to describe the process by which the disclosed compositions may be delivered to a subject. Administration will often depend upon the amount of composition administered, the number of doses, and duration of treatment. Multiple doses of the composition may be administered. The frequency of administration of the composition can vary depending on any of a variety of factors, such as level blood glucose before administration, and the like. The duration of administration of the composition, e.g., the period of time over which the composition is administered, can vary, depending on any of a variety of factors, including patient response, etc. The composition may be administered in various ways including oral, intragastric, and parenteral (referring to intravenous and intra-arterial and other appropriate parenteral routes).

The amount of the composition administered can vary according to factors such as the degree of susceptibility of the individual, the age, sex, and weight of the individual, idiosyncratic responses of the individual, the dosimetry, and the like. Detectably effective amounts of the composition of the present disclosure can also vary according to instrument and film-related factors. Optimization of such factors is well within the level of skill in the art.

As used herein “beta-hydroxybutyrate,”, “βHB”, or “BHB” as used interchangeably herein refer to a carboxylic acid having the general formula CH₃CH₂OHCH₂COOH. BHB is a ketone body which may be utilized by the body as a fuel source during instances of low glucose levels.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

“Derivative” refers to a compound or portion of a compound that is derived from or is theoretically derivable from a parent compound.

The term “hydroxyl group” is represented by the formula —OH.

The term “alkoxy group” is represented by the formula —OR, where R can be an alkyl group, including a lower alkyl group, optionally substituted with an alkenyl, alkynyl, aryl, aralkyl, cycloalkyl, halogenated alkyl, or heterocycloalkyl group, as defined below.

The term “ester” as used herein is represented by the formula —OC(O)R, where R can be an alkyl, alkenyl, alkynyl, aryl, aralkyl, cycloalkyl, halogenated alkyl, or heterocycloalkyl group, as defined below.

The term “alkyl group” is defined as a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. A “lower alkyl” group is a saturated branched or unbranched hydrocarbon having from 1 to 10 carbon atoms.

The term “alkenyl group” is defined as a hydrocarbon group of 2 to 24 carbon atoms and structural formula containing at least one carbon-carbon double bond.

The term “alkynyl group” is defined as a hydrocarbon group of 2 to 24 carbon atoms and a structural formula containing at least one carbon-carbon triple bond.

The term “halogenated alkyl group” is defined as an alkyl group as defined above with one or more hydrogen atoms present on these groups substituted with a halogen (F, Cl, Br, I).

The term “cycloalkyl group” is defined as a non-aromatic carbon-based ring composed of at least three carbon atoms. Examples of cycloalkyl groups include, but are not limited to, cyclopropyl, cyclobutyl, cyclopentyl, cyclohexyl, etc.

The term “heterocycloalkyl group” is a cycloalkyl group as defined above where at least one of the carbon atoms of the ring is substituted with a heteroatom such as, but not limited to, nitrogen, oxygen, sulfur, or phosphorous.

The term “aliphatic group” is defined as including alkyl, alkenyl, alkynyl, halogenated alkyl and cycloalkyl groups as defined above. A “lower aliphatic group” is an aliphatic group that contains from 1 to 10 carbon atoms.

The term “aryl group” is defined as any carbon-based aromatic group including, but not limited to, benzene, naphthalene, etc. The term “aromatic” also includes “heteroaryl group,” which is defined as an aromatic group that has at least one heteroatom incorporated within the ring of the aromatic group. Examples of heteroatoms include, but are not limited to, nitrogen, oxygen, sulfur, and phosphorous. The aryl group can be substituted with one or more groups including, but not limited to, alkyl, alkynyl, alkenyl, aryl, halide, nitro, amino, ester, ketone, aldehyde, hydroxy, carboxylic acid, or alkoxy, or the aryl group can be unsubstituted.

The term “aralkyl” is defined as an aryl group having an alkyl group, as defined above, attached to the aryl group. An example of an aralkyl group is a benzyl group.

“Esterification” refers to the reaction of an alcohol with a carboxylic acid or a carboxylic acid derivative to give an ester.

“Transesterification” refers to the reaction of an ester with an alcohol to form a new ester compound.

“Ketogenic compound” refers a compound that is capable of elevating ketone body concentrations in a subject.

“Ketone” or “ketone body”, as used interchangeably herein, refers to a compound or species which is β-hydroxybutyrate (βHB), acetoacetate, acetone, or a combination thereof. A ketone body may be derived from a ketone body precursor, that is, a compound or species which is a precursor to a ketone body and which may be converted or metabolized to a ketone body in a subject.

“Ketone body ester” or “ketone ester” as used herein, refer to an ester of a ketone body, ketone body precursor, or derivative thereof. Any suitable ketone ester known in the art may be used. For example, the ketone ester may be 1,3 butanediol acetoacetate diester.

“Ketone body salt” or “ketone salt” is a salt of a ketone body, ketone body precursor, or derivative thereof. The ketone salt may be combined with a monovalent cation, divalent cation, or alkaline amino acid. Any suitable ketone salt known in the art may be used. For example, the ketone salt may be a BHB salt. The ketone salt may be a BHB mineral salt. For example, the BHB mineral salt may be potassium βHB, sodium βHB, calcium βHB, magnesium βHB, lithium BHB, or any other feasible non-toxic mineral salts of βHB. The ketone salt may be a BHB organic salt. Organic salts of BHB include salts of organic bases such as arginine βHB, lysine βHB, histidine BHB, ornithine βHB, creatine βHB, agmatine βHB, and citrulline βHB. The ketone salt may be a combination of any of the BHB salts. For example, the ketone salt may be sodium beta-hydroxybutyrate and arginine beta-hydroxybutyrate, or beta-hydroxybutyrate sodium salt and beta-hydroxybutyrate potassium salt.

“Ketosis”, “ketogenic state”, or “nutritional ketosis” as used interchangeably herein refers to a subject having blood ketone levels within the range of about 0.5 mmol/L to about 10 mmol/L. Nutritional ketosis as used herein is distinguished from diabetic or alcoholic ketoacidosis. Alcoholic ketoacidosis is associated with an excessive accumulation of blood ketone body levels and a drop in blood pH. Diabetic ketoacidosis is associated with, for example, the absence of insulin, blood ketone levels in excess of 10 mmol/L, metabolic derangement, and electrolyte imbalance.

“Keto-adaptation” as used herein refers to prolonged nutritional ketosis (>1 week) to achieve a sustained non-pathological ketosis.

“Ketogenic diet” as used herein refers to a diet induces a metabolic switch from burning glucose for energy to burning fats for energy. A ketogenic state may be achieved through calorie restriction, fasting, prolonged exercise, and/or a ketogenic diet that is high in fat and restricted in sugars and starchy carbohydrates.

The term “medium chain triglycerides” (MCT) as used herein refers to molecules having a glycerol backbone attached to three medium chain fatty acids. Medium chain fatty acids range from 6 to 12 carbon atoms in length. Exemplary fatty acids are caprylic acid, also known as octanoic acid, comprising 8 carbon molecules, and capric acid, also known as decanoic acid, comprising 10 carbon molecules.

A “therapeutically effective amount,” or “effective dosage” or “effective amount” as used interchangeably herein unless otherwise defined, means a dosage of a drug effective for periods of time necessary, to achieve the desired therapeutic result. An effective dosage may be determined by a person skilled in the art and may vary according to factors such as the disease state, age, sex, and weight of the individual, and the ability of the drug to elicit a desired response in the individual. The effective dosage will depend on absorption, distribution, metabolism, and excretion rates of the disclosed exogenous ketones. The dose should be sufficient to affect a desirable response, such as reducing blood glucose levels in the subject.

A therapeutically effective amount may be administered in one or more administrations (e.g., the composition may be given as a preventative treatment or therapeutically at any stage of disease progression, before or after symptoms, and the like), applications or dosages and is not intended to be limited to a particular formulation, combination or administration route. It is within the scope of the present disclosure that the disclosed ketogenic compound may be administered at various times during the course of treatment of the subject. The times of administration and dosages used will depend on several factors, such as the goal of treatment (e.g., treating v. preventing), condition of the subject, etc. and can be readily determined by one skilled in the art. Administration may be adjusted according to individual need and professional judgment of a person administrating or supervising the administration of the compounds used in the present invention.

A “pharmaceutically acceptable excipient,” “pharmaceutically acceptable diluent,” “pharmaceutically acceptable carrier,” or “pharmaceutically acceptable adjuvant” as used herein means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use, such as those promulgated by the United States Food and Drug Administration.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). The subject may be a human or a non-human. The subject or patient may be undergoing other forms of treatment.

### 2. METHODS OF REDUCING BLOOD GLUCOSE

The disclosure provides methods for reducing blood glucose levels in a subject. The method may comprise administering to the subject a composition comprising one or more ketogenic compounds. The ketogenic compound may be any compound capable of elevating ketone body concentrations in a subject. For example, the ketogenic compound may elevate expression of BHB following administration to the subject. The ketogenic compound may be a ketone body precursor or derivative thereof. Any suitable ketone body precursor which will be metabolized into a ketone body upon administration to the subject may be used. For example, the ketogenic compound may comprise any one or more of 1,3-butanediol, acetoacetate, or BHB moieties or derivatives thereof, including esters and salts thereof. For example, the ketogenic compound may be 1,3-butanediol, ethyl acetoacetate, or ethyl BHB.

The ketogenic compound may be a ketone ester. Any suitable ketone ester may be used in the disclosed composition. Ketone esters may be prepared using any suitable physiologically compatible alcohol. Examples of polyhydric alcohols suitable for preparing such esters include carbohydrates and carbohydrate derivatives, such as carbohydrate alcohols. Examples of carbohydrates include, without limitation, altrose, arabinose, dextrose, erythrose, fructose, galactose, glucose, gulose, idose, lactose, lyxose, mannose, ribose, sucrose, talose, threose, xylose and the like. The ketone ester may be a monoester. The ketone ester may be a diester, The ketone ester may be a polyester. Exemplary ketone esters include 1,3-butanediol-acetoacetate monoester and 1,3-butanediol-acetoacetate diester, synthesized as previously described (Agostino et al., Am J Physiol; 304(10): R829-R836 (2013)).

The ketogenic compound may comprise a ketone salt. The ketone salt may be combined with a monovalent cation, divalent cation, or alkaline amino acid. Any suitable ketone salt may be used. For example, the ketone salt may be a BHB salt. The ketone salt may be a BHB mineral salt. For example, the BHB mineral salt may be potassium βHB, sodium βHB, calcium βHB, magnesium βHB, lithium BHB, or any other feasible non-toxic mineral salts of βHB. The ketone salt may be a BHB organic salt. Organic salts of BHB include salts of organic bases such as arginine βHB, lysine βHB, histidine BHB, ornithine βHB, creatine βHB, agmatine βHB, and citrulline βHB. The ketone salt may be a combination of BHB salts. For example, the ketone salt may be a sodium/potassium BHB mineral salt.

The ketone salt may be mixed into a solution. For example, a βHB mineral salt may be mixed into a solution. The βHB mineral salt may be from 5% to 60% of a solution. For example, the βHB mineral salt may be 5%, may be 6%, may be 7%, may be 8%, may be 9%, may be 10%, may be 11%, may be 12%, may be 13%, may be 14%, may be 15%, may be 16%, may be 17%, may be 18%, may be 19%, may be 20%, may be 21%, may be 22%, may be 23%, may be 24%, may be 25%, may be 26%, may be 27%, may be 28%, may be 29%, may be 30%, may be 31%, may be 32%, may be 33%, may be 34%, may be 35%, may be 36%, may be 37%, may be 38%, may be 39%, may be 40%, may be 41%, may be 42%, may be 43%, may be 44%, may be 45%, may be 46%, may be 47%, may be 48%, may be 49%, may be 50%, may be 51%, may be 52%, may be 53%, may be 54%, may be 55%, may be 56%, may be 57%, may be 58%, may be 59%, or may be 60%. Preferably, the βHB mineral salt is about 50% of a solution. In an embodiment, the βHB mineral salt is comprised of about 375 mg/g of pure βHB and about 125 mg/g of Na⁺/K⁺. The dose of the ketone salt may be from about 1000 mg to about 25000 mg of βHB, depending on the weight of the subject. For example, the dose of the βHB mineral salt may be from about 1100 mg to about 24,000 mg, about 1200 mg to about 23,000 mg, about 1300 mg to about 22,000 mg, about 1400 mg to about 21,000 mg, about 1500 mg to about 20,000 mg, about 1600 mg to about 19,000 mg, about 1700 mg to about 18,000 mg, about 1800 mg to about 17,000 mg, about 1900 mg to about 16,000 mg, about 2000 mg to about 15,000 mg, about 2100 mg to about 14,000 mg, about 2200 mg to about 13,000 mg, about 2300 mg to about 12,000 mg, about 2400 mg to about 11,000 mg, about 2500 mg to about 10,000 mg, about 2600 mg to about 9000 mg, about 2700 mg to about 8000 mg, about 2800 mg to about 7000 mg, about 2900 mg to about 6000 mg, about 3000 mg to about 5000 mg, or about 3100 mg to about 4000 mg. For example, the dose may be about 1000 mg, about 1100 mg, about 1200 mg, about 1300 mg, about 1400 mg, about 1500 mg, about 1600 mg, about 1700 mg, about 1800 mg, about 1900 mg, about 2000 mg, about 3000 mg, about 4000 mg, about 5000 mg, about 6000 mg, about 7000 mg, about 8000 mg, about 9000 mg, about 10,000 mg, about 11,000 mg, about 12,000 mg, about 13,000 mg, about 14,000 mg, about 15,000 mg, about 16,000 mg, about 17,000 mg, about 18,000 mg, about 19,000 mg, about 20,000 mg, about 21,000 mg, about 22,000 mg, about 23,000, mg, about 24,000 mg, or about 25,000 mg.

The composition may additionally comprise at least one medium chain fatty acid or ester thereof. For example, the composition may additionally comprise at least one medium chain triglyceride. The composition may comprise MCT oil. Sources of the medium chain fatty acid or an ester thereof include coconut oil, coconut milk powder, fractionated coconut oil, palm oil, palm kernel oil, caprylic acid, isolated medium chain fatty acids such as isolated hexanoic acid, isolated octanoic acid, isolated decanoic acid, medium chain triglycerides either purified or in natural form such as coconut oil, and ester derivatives of the medium chain fatty acids ethoxyiated triglyceride, enone triglyceride derivatives, aldehyde triglyceride derivatives, monoglyceride derivatives, diglyceride derivatives, and triglyceride derivatives, and salts of the medium chain triglycerides. Ester derivatives optionally include alkyl ester derivatives, such as methyl, ethyl, propyl, butyl, hexyl, etc. Derivatives may be prepared by any process known in the art, such as direct esterification, rearrangement, fractionation, transesterification, or the like.

The disclosed composition may comprise any combination of one or more ketogenic compounds. For example, the disclosed composition may comprise a combination of any one or more of a ketone ester, a ketone salt, a ketone body precursor, and a medium chain fatty acid. The composition may comprise at least one ketone salt and at least one ketone ester. For example, the composition may comprise sodium/potassium BHB mineral salt and 1,3 butanediol acetoacetate diester. The composition may comprise at least one ketone salt and at least one medium chain fatty acid. For example, the composition may comprise sodium/potassium BHB mineral salt a MCT. The composition may comprise at least one ketone ester and at least one medium chain fatty acid. For example, the composition may comprise 1,3 butanediol acetoacetate diester a MCT. The composition may comprise a ketone salt and a MCT mixed at an approximate 1:1 ratio. The composition may comprise a ketone ester and a MCT mixed at an approximate 1:1 ratio. The composition may comprise a ketone precursor and a MCT mixed at an approximate 1:1 ratio. The above combinations are intended strictly to provide examples and are in no way limiting to other combinations that may be used.

The composition may additionally comprise other nutritional substrates. For example, the composition may additionally free amino acids, amino acid metabolites, vitamins, minerals, electrolytes and metabolic optimizers such as NADH, soluble ubiquinol, tetrahydrobiopeterin, alpha-ketoglutaric acid, carnitine, and/or alpha lipoic acid, nutritional co-factors, calcium beta-methyl-beta-hydroxybutyrate, arginine alpha-ketoglutarate, sodium R-alpha lipoic acid, thiamine, riboflavin, niacin, pyridoxine, ascorbic acid, citric acid, malic acid, sodium benzoate, potassium sorbate, acesulfame K, aspartame, xanthan gum, or a combination thereof. Non-limiting examples of nutritional co-factors include R-alpha lipoic acid, acetyl-1-carnitine, ketoisocaproate, alpha-ketoglutarate, alpha-hydroxyisocaproate, creatine, branched chain amino acids (leucine, isoleucine, valine), beta-hydroxy-beta methylbutyrate (HMB), B vitamins, vitamin C, soluble ubiquinol, and carnitine that assist in mitochondrial function.

The composition may be delivered to the subject in any dose sufficient to achieve the desired therapeutic effect, e.g. reduction of blood glucose levels in the subject. For example, the composition may be administered in a dosage range of 1 mg ketogenic compound/kg of body weight to 100 g ketogenic compound/kg body weight. A therapeutically effective amount of a ketogenic compound of the disclosed composition may be about 1 mg ketogenic compound/kg body weight to about 25,000 mg/kg, about 5 mg/kg to about 10,000 mg/kg, about 10 mg/kg to about 5,000 mg/kg, about 15 mg/kg to about 1,000 mg/kg, about 20 mg/kg to about 800 mg/kg, about 25 mg/kg to about 750 mg/kg, about 30 mg/kg to about 700 mg/kg, about 35 mg/kg to about 650 mg/kg, about 40 mg/kg to about 600 mg/kg, about 45 mg/kg to about 550 mg/kg, about 50 mg/kg to about 500 mg/kg, about 55 mg/kg to about 450 mg/kg, about 60 mg/kg to about 400 mg/kg, about 65 mg/kg to about 350 mg/kg, about 70 mg/kg to about 300 mg/kg, about 75 mg/kg to about 250 mg/kg, about 80 mg/kg to about 200 mg/kg, about 85 mg/kg to about 150 mg/kg, and about 90 mg/kg to about 100 mg/kg. A therapeutically effective amount of a ketogenic compound of the disclosed composition may be about 1.25 mg/kg, about 2.5 mg/kg, about 5 mg/kg, or about 10 mg/kg. A therapeutically effective amount of a ketogenic compound of the disclosed composition may be about 1.25 g/kg, about 2.5 g/kg, about 5 g/kg, about 10 g/kg, or about 25 g/kg.

The composition may be administered in various ways, including, for example, orally, intragastricly, or parenterally (referring to intravenously and intra-arterially and other appropriate parenteral routes), among others. Administration may be as a single dose, or multiple doses over a period of time. Administration may be as a single dose, or multiple doses over a period of time. In an embodiment, the composition may be administered chronically, for example, between about 1 day and about 7 days), or sub-chronically (e.g., more than 7 days). For example, multiple doses may be delivered over 1 day, 3 days, 5 days, 7 days, 10 days, 14 days, or more, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more, 1 month, 2 months, 3 months, 4 months, 5 months, 6 months, 7 months, 8 months, 9 months, 10 months, 11 months, 12 months, or more.

The composition may be a solid, for example a powder, tablet, gel, bar, confectionary product, or a granule, and intended for use as a solid oral dose form. The solid composition may be mixed before use with a liquid, such as a water-based liquid (e.g., fruit drink, dairy product, milk, and yogurt), to provide a liquid drink for the user. The composition may be provided, as desired, as a liquid product in a form ready for consumption or as a concentrate or paste suitable for dilution on use. The liquid product may be pH adjusted with citric and/or malic acid, and artificial sweetener and flavoring can be added. The liquid product may be homogenized and pasteurized. The composition may further comprise a pharmaceutically acceptable excipient, diluent, or carrier.

The levels of circulating glucose and ketone bodies may be measured in a subject prior to or following administration of the disclosed composition. Circulating levels may be determined from, for example, bodily fluids (e.g. blood, serum, plasma, or urine) or breath (such as, acetone on the breath). Any suitable measuring device or kit known in the art may be used, such as the PRECISION XTRA® blood glucose and ketone monitoring kit (Abbott Laboratories, Abbott Park, Ill.).

The invention further discloses a kit, which may be used to induce ketosis in a subject. Instructions included in kits may be affixed to packaging material or may be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### 3. EXAMPLES

The disclosed compounds, compositions, processes, and methods will be better understood by reference to the following examples, which are intended as an illustration of and not a limitation upon the scope of the invention. Where the term comprising is used herein, it should be understood that the disclosure also contemplates alternative embodiments consisting of or consisting essentially of the recited features.

### Example 1—Methods

Animals—

Male Sprague-Dawley Rats (SPD), male WAG/Rij rats, and male G1D mice were used in the following experiments. The animals were housed at Department of Molecular Pharmacology and Physiology (Hyperbaric Biomedical Research Laboratory, Morsani College of Medicine, University of South Florida, Tampa, Fla., USA) and the Department of Zoology (University of West Hungary, Savaria Campus, Szombathely, Hungary). Animals were kept in groups of 2-4 under standard laboratory conditions (12:12 h light-dark cycle, light was on from 08:00 AM to 08:00 PM) in air-conditioned rooms at 22±2° C.

Animal treatment and measuring procedures were performed in accordance with the University of South Florida Institutional Animal Care and Use Committee (IACUC) guidelines (Protocol #0006R) and with the local ethical rules in accordance with the Hungarian Act of Animal Care and Experimentation (1998. XXVIII. Section 243/1998) in conformity with the regulations for animal experimentation in the European Communities Council Directive of 24 Nov. 1986 (86/609/EEC). All experiments were approved by the University of South Florida IACUC and all efforts were made to reduce the number of animals used.

Synthesis and Formulation of Ketogenic Compounds—

Ketone ester (KE; 1,3-butanediol-acetoacetate diester) was synthesized as previously described (Agostino et al., Am J Physiol; 304(10): R829-R836 (2013). The ketone salt Na⁺/K⁺-β-hydroxybutyrate mineral salt (KS) is a novel agent that was mixed into a 50% solution supplying approximately 375 mg/g of pure βHB and 125 mg/g of Na⁺/K⁺ in a 1:1 ratio. Both KE and KS were developed and synthesized in collaboration with Savind Inc. Human food grade medium chain triglyceride (MCT) oil (˜60% caprylic triglyceride/40% capric triglyceride) was purchased from Now Foods (Bloomingdale, Ill., USA). KS was mixed with MCT in a 1:1 ratio (KSMCT) at the University of South Florida (USF, Tampa, Fla., USA).

Statistics—

All data are presented as the mean±standard error of the mean (SEM). The effects of ketogenic compounds on anxiety level, as well as on blood βHB and glucose levels, was compared to control or/and baseline levels. Data analysis was performed using GraphPad PRISM version 6.0a. Results were considered significant when *p<0.05, **p<0.005, ***p<0.0005. Significance was determined by unpaired t-test. Blood ketone, and blood glucose levels were compared using a two-way ANOVA with Tukey's multiple comparisons test and unpaired t-test.

### Example 2—Glucose Reduction without Exercise in Sprague-Dawley and WAG/Rij Rats

The ability of the disclosed ketogenic compounds to decrease blood glucose levels without exercise was tested in rats.

Chronic Administration—

A total of 48 male SPD rats were fed for 83 days with either standard rodent chow (2018 Teklad Global 18% Protein Rodent Diet (#2018), Harlan) standard diet (SD)/control; n=9) or SD+ketone supplementation. Four treatment animal groups included low-dose KE (˜10 g/kg b.w./day, Low-dose ketone ester (LKE); n=10), high-dose KE (˜25 g/kg b.w./day, high dose ketone ester (HKE); n=10), KS (˜25 g/kg b.w./day, KS; n=9) and KSMCT (˜25 g/kg b.w./day, KSMCT; n=10).

Sub-Chronic Oral Gavage—

In order to familiarize the animals to the intragastric gavage method, water was gavaged for 5 days before ketone supplementation. Following the adaptation period to the intragastric gavage method, 39 male SPD rats were fed with standard diet, and gavaged daily with 5 g/kg b.w./day water (SD/control; n=11) or ketone supplements KE (n=9), KS (n=9), KSMCT (n=10) sub-chronically for 7 days. In addition, following the adaptation period to the intragastric gavage method, WAG/Rij male rats (n=32) were fed with SD and gavaged sub-chronically with about 2.5 g/kg b.w./day water (SD/control; n=8), KE (n=8), KS (n=8) or KSMCT (n=8) for 7 days.

Measurement of Blood βHB and Glucose—

In the chronic feeding study, blood βHB and glucose levels were measured 24 h before the 1st day of ketone treatments (baseline levels) and at 13th week. In the 7 day oral gavage studies, blood βHB and glucose levels were measured 24 h before the 1st day of ketone treatments (baseline levels; SPD and WAG/Rij rats), 24 h after the first gavage and 60 min after gavage on the 7th day (SPD and WAG/Rij rats). Whole blood samples (10 μL) were taken from the saphenous vein for analysis of blood glucose (mg/dl) and βHB (mmol/1) levels with the commercially available glucose and ketone (βHB) monitoring system Precision Xtra™ (Abbott Laboratories, Abbott Park, Ill., USA).

Elevation of Blood βHB Levels in SPD Rats with Ketone Supplements—

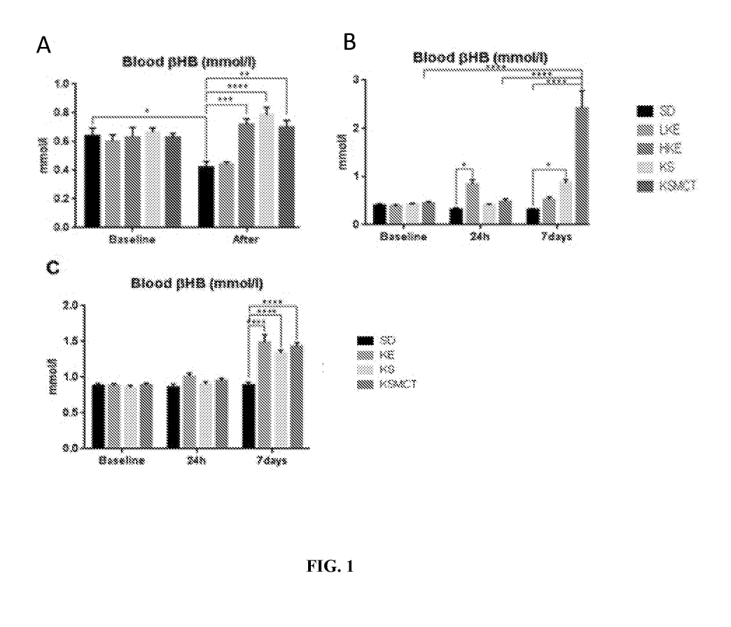

After 83 days of chronic feeding in SPD rats, blood βHB levels remained significantly elevated in HKE, KS and KSMCT groups compared to control while it decreased in SD compared to baseline (FIG. 1A). Blood βHB levels were elevated after 24 h of a single gavage in KE group compared to control. βHB was elevated in KSMCT groups at 7 days compared to their level at 24 h and baseline. Blood βHB was also elevated in KS and KSMCT treatment groups compared to control group (FIG. 1B).

Elevation of Blood βHB Levels in Wag/Rij Rats with Ketone Supplements—

After 7 days of gavage, blood βHB was elevated in KE, KS and KSMCT groups in WAG/Rij rats compared to baseline, 24 h and control (FIG. 1C).

Ketone Supplementation and Blood Glucose Levels in SPD Rats—

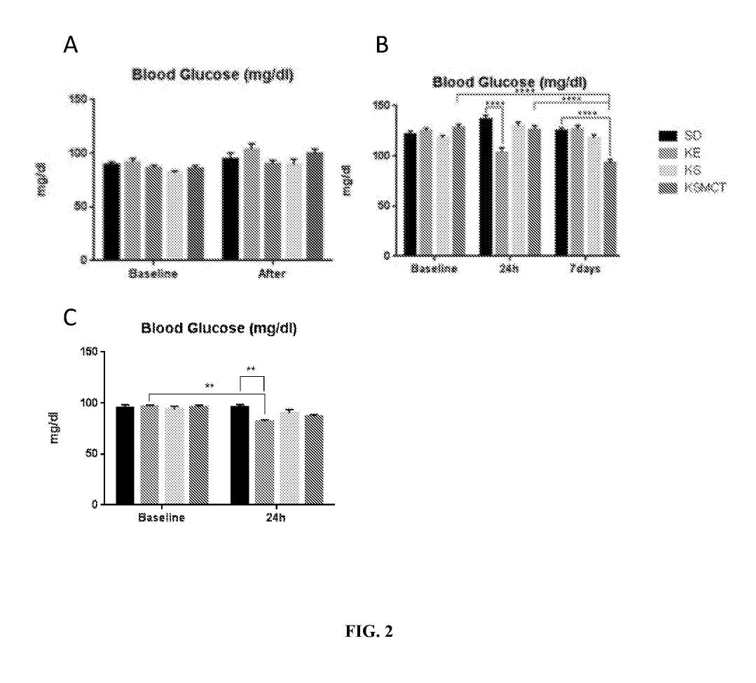

After 13 weeks of chronic feeding in SPD rats blood glucose did not change significantly in any groups (FIG. 2A). After sub-chronic ketone treatments, blood glucose levels were lower at 24 h in KE group compared to control. After 7 days of oral gavage blood glucose was lower in KSMCT compared to control, to baseline and to the level at 24 h in SPD rats (FIG. 2B).

Ketone Supplementation and Blood Glucose Levels in WAG/Rij Rats—

In WAG/Rij rats the KE group had lower glucose levels after 24 h, compared to baseline levels and compared to control group. (FIG. 2C).

### Example 3—Glucose Reduction Following Multiple Treatment and Exercise in Sprague-Dawley Rats

The ability of the disclosed compositions to reduce blood glucose levels following multiple treatments and exercise was tested in Sprague-Dawley rats. Four month old Sprague-Dawley rats were first acclimated to oral gavage with administration of tap water once daily for a period of 5 days. The rats were then divided into 5 groups, and fed either a standard diet (SD), a ketogenic diet (KD), or a standard diet supplemented with a once daily oral gavage of a ketogenic compound as outlined in Table 1. The ketogenic compound was selected from 1,3-butanediol-acetoacetate diester (SD+KE), β-hydroxybutyrate mineral salt (SD+KS), or β-hydroxybutyrate mineral salt and a medium chain triglyceride (SD+KSMCT). The above ketogenic compounds were provided at a dose of 5 g/kg of body weight.

TABLE 1

| | Ketogenic | | | |
|---|---|---|---|---|
| Diet | Compound | Details | n rats | Abbreviation |
| Standard Diet | — | — | 11 | SD |
| Ketogenic Diet | — | — | 10 | KD |
| Standard Diet | Ketone ester | 1,3-butanediol- | 9 | SD + KE |
| | | acetoacetate | | |
| | | diester | | |
| Standard Diet | Ketone salt | β- | 9 | SD + KS |
| | | hydroxybutyrate | | |
| | | mineral salt | | |
| Standard Diet | Ketone salt | β- | 10 | SD + KSMCT |
| | and fatty | hydroxybutyrate | | |
| | acids | mineral salt and | | |
| | | a medium chain | | |
| | | triglyceride | | |
| | | (MCT) | | |

Each group of animals was maintained on the respective dietary conditions for 7 days. Whole blood samples (10 μL) were collected from the saphenous vein of rats. The levels of blood glucose (mg/dL) and β-hydroxybutyrate (mmol/L) were measured with the commercially available glucose monitoring system PRECISION XTRA® (Abbott Laboratories, Abbott Park, Ill., USA). It is noted that the PRECISION XTRA® only measures β-hydroxybutyrate levels, therefore total blood ketone levels may be higher than measured.

Rats were exercised on a Rota Rod Rotamex 5 (Columbus Instruments). Animals were exercised on the rotarod for 5 consecutive days before treatment began in order to acclimate to the equipment and the task. The rats were placed on rods of the accelerating rotarod and the time the animals remained on the rotarod was measured. The rotarod was set to accelerate to 40 rpm in 180 seconds. Each training and testing consisted of 3 trials, with a 2 minute rest period between each trial. Animals were placed on the rotarod and timed until they fell from the rotating rod. The animals were evaluated on the rotarod exercise test after the 5 days training period but prior to the beginning of dietary treatment (baseline), 24 hours after treatment, and after 7 days of dietary treatment.

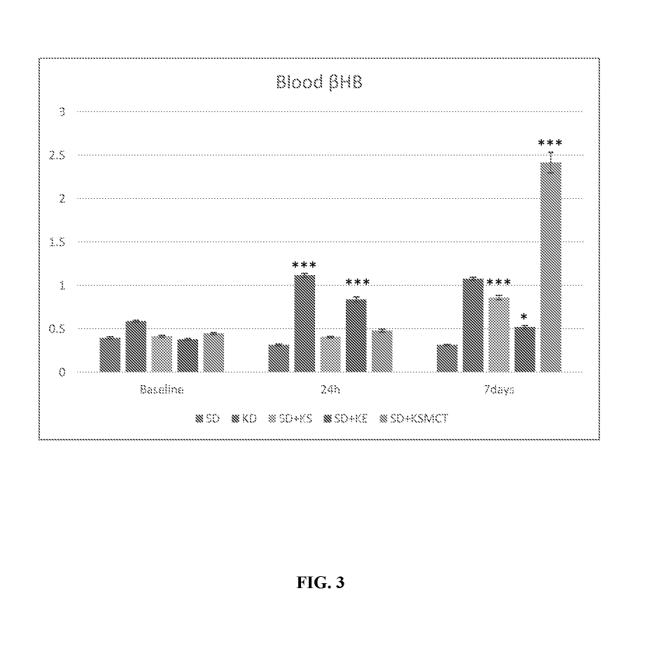

The animals were evaluated for blood levels of glucose and ketone bodies β-hydroxybutyrate), prior to the beginning of dietary treatment, 24 hours after treatment, and after 7 days of dietary treatment. Blood levels of BHB after exercise are shown in FIG. 3. Rats fed a ketogenic diet exhibited an increase in βHB at both 24 hours and 7 days following treatment, while rats given a standard diet supplemented with a ketone ester only showed elevated levels by 24 hours and a trend towards baseline levels by 7 days. Rats fed a standard diet supplemented with ketone salt, or ketone salt with MCT, showed an opposing trend, with no change in βHB at 24 hours despite a significant increase at 7 days.

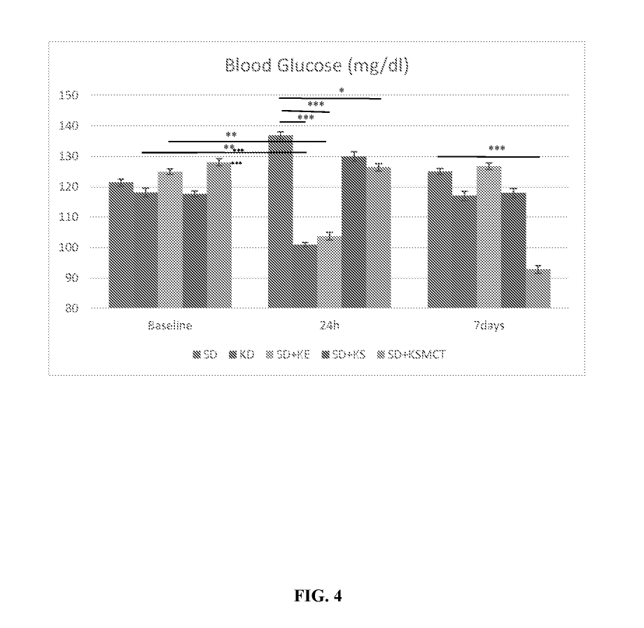

Blood glucose levels after exercise are shown in FIG. 4. Rats fed a ketogenic diet showed decreased blood glucose levels after 24 hours compared to baseline. Rats fed a standard diet supplemented with a ketone ester also showed reduce blood glucose levels after 24 hours. Levels trended towards baseline after 7 days. Rats supplemented with a ketone salt showed an increase in blood glucose by 24 hours, with a decline towards baseline by 7 days. Rats given ketone salt and MCT showed no changes in glucose after 24 hours, but significant decreases in glucose levels after 7 days.

### Example 4—Glucose Reduction Following Single Treatment and Exercise in Sprague-Dawley Rats

The ability of the disclosed compositions to reduce blood glucose levels following a single treatment and exercise was tested in one year old Sprague-Dawley rats.

Rats were divided into 6 groups, and treated with a standard diet (SD) or a SD supplemented with a once daily oral gavage of a ketogenic compound. The ketogenic compound was selected from 1, 3-butanediol (BD), β-hydroxybutyrate mineral salt with a medium chain triglyceride (KS+MCT), 1,3-butanediol-acetoacetate diester with β-hydroxybutyrate mineral salt (KE+KS), 1,3-butanediol-acetoacetate diester with a medium chain triglyceride (KE+KSMCT), and 1,3-butanediol-acetoacetate diester (KE).

Rats were exercised on a Rota Rod Rotamex 5 (Columbus Instruments). Animals were trained on the rotarod for 5 consecutive days before treatment begin in order to acclimate to the equipment and the task. The rats were placed on rods of the accelerating rotarod and the time the animals remained on the rotarod was measured. The rotarod was set to accelerate to 40 rpm in 180 seconds. Each training and testing consisted of 3 trials, with a 2 minute rest period between each trial. Animals were placed on the rotarod and timed until they fell from the rotating rod. The animals were evaluated on the rotarod exercise test after the 5 days training period but prior to the beginning of dietary treatment (baseline). On the next day the animals received gavage treatment and were evaluated on the rotarod exercise test 30-45 min after treatment.

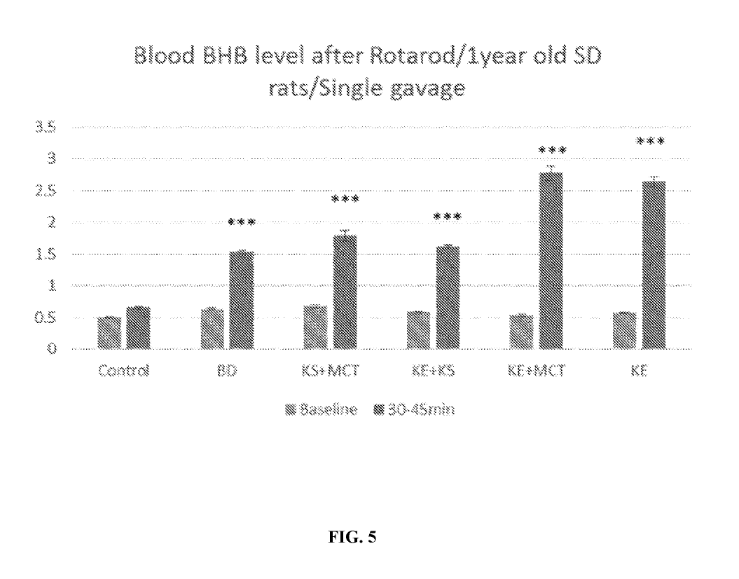

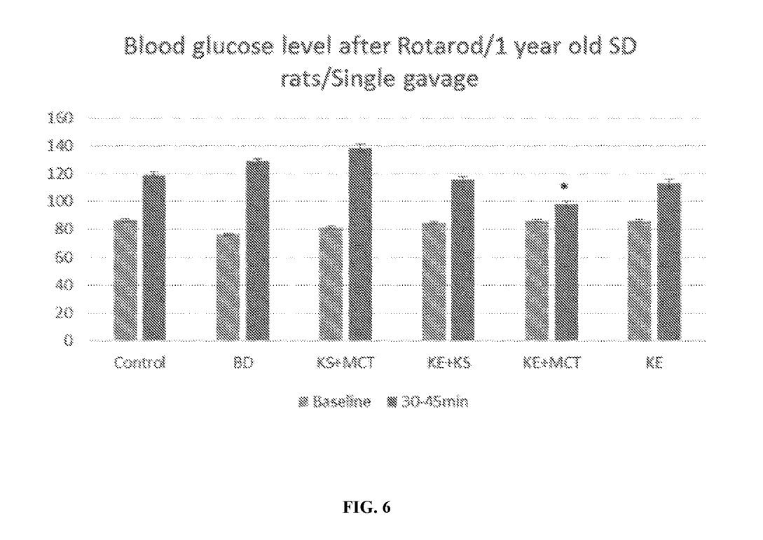

Rats showed significantly higher levels of blood βHB 30-45 minutes after oral gavage in all treatment groups compared to the control group following exercise (FIG. 5). Rats showed significant increases in blood glucose levels with exercise 30-45 minutes after oral gavage in all treatment groups compared to the control group with the exception of the ketone ester with MCT group. The ketone ester with MCT group showed a significantly lower blood glucose level compared to control group following exercise (FIG. 6). These results demonstrate that a combination of a ketone ester and MCT prevents increased levels of blood glucose following exercise.

### Example 5—Glucose Reduction Following Single Treatment in WAG/Rij Rats without Exercise

The ability of the disclosed compositions to reduce blood glucose levels was subsequently tested in WAG/Rij rats. WAG/Rij rats are used as a genetic model of absence epilepsy with comorbidity of depression and anxiety.

WAG/Rij rats were first acclimated to oral gavage with administration of tap water once daily for a period of 3 days. The mice were then divided into 6 groups, and given an oral gavage supplement selected from water, 1,3-butanediol-acetoacetate diester (KE), β-hydroxybutyrate mineral salt (KS), 1,3-butanediol-acetoacetate diester with medium chain triglyceride (KEMCT), β-hydroxybutyrate mineral salt with medium chain triglyceride (KSMCT), or a combination of 1,3-butanediol-acetoacetate diester and β-hydroxybutyrate mineral salt (KEKS). Compositions containing 1,3-butanediol-acetoacetate diester or β-hydroxybutyrate mineral salt alone were provided at a dose of 2.5 g/kg of body weight. The compositions containing two components (e.g., 1,3-butanediol-acetoacetate diester with MCT, β-hydroxybutyrate mineral salt with MCT, 1,3-butanediol-acetoacetate diester with β-hydroxybutyrate mineral salt), each component was provided at a dose of 1.25 g/kg of body weight (for a dosage of 2.5 g/kg for 2 components). The animals were evaluated for blood levels of glucose and β-hydroxybutyrate, before administration of the composition, and at 60 minutes after administration.

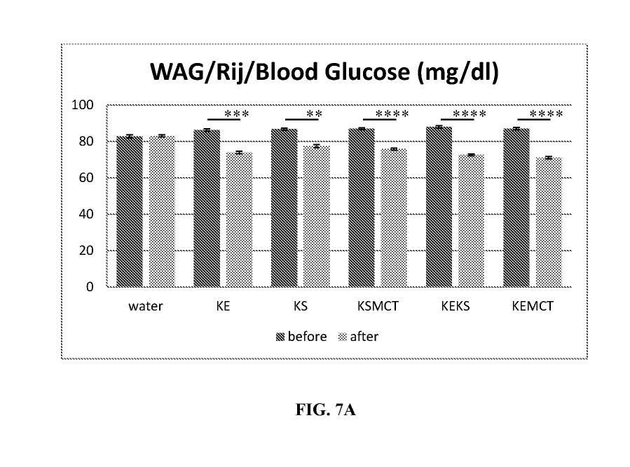

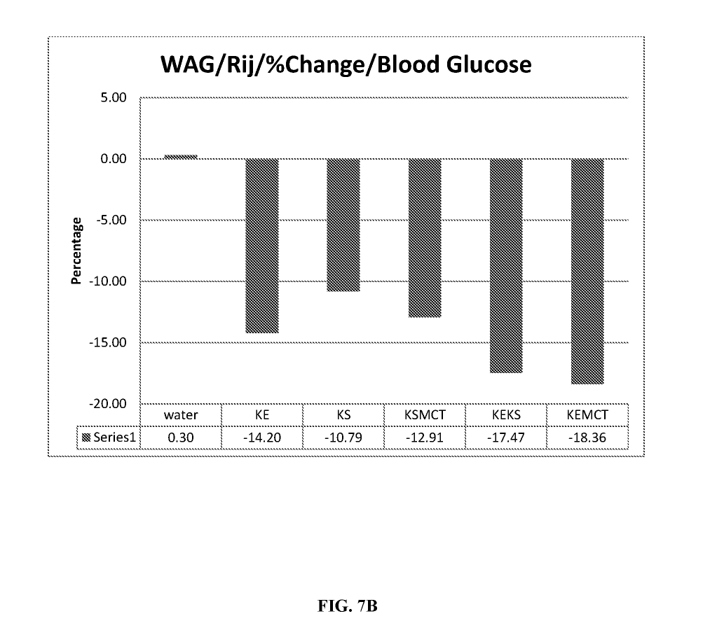

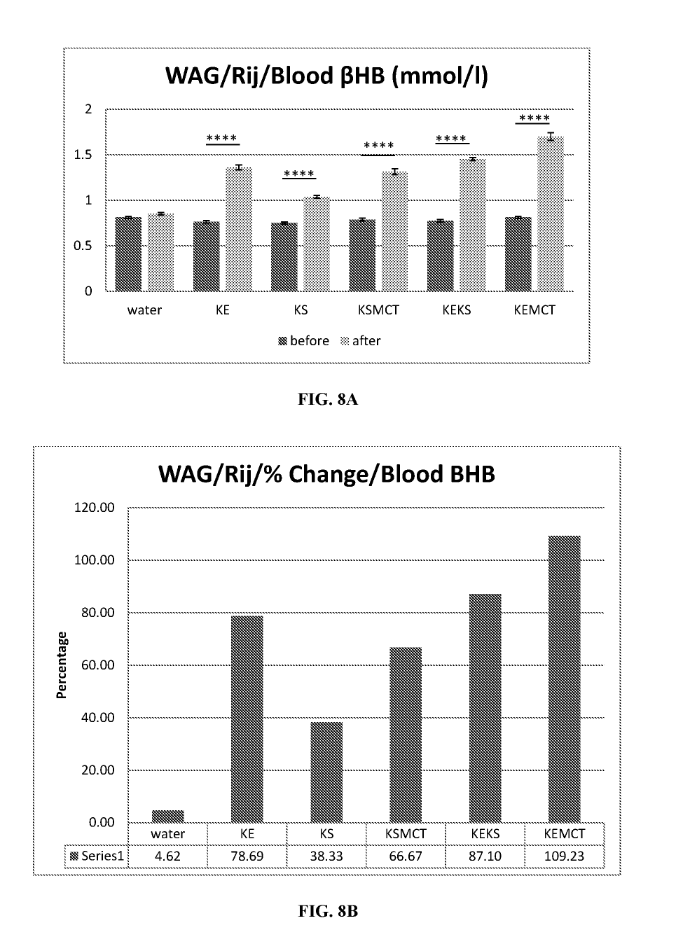

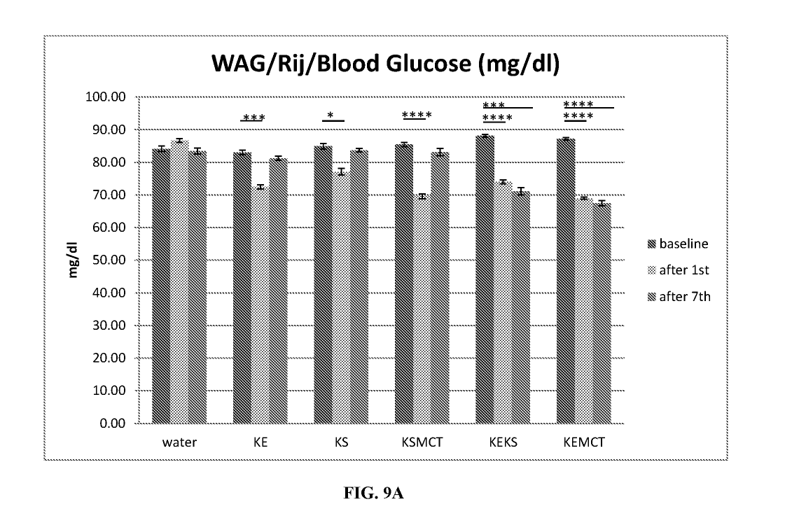

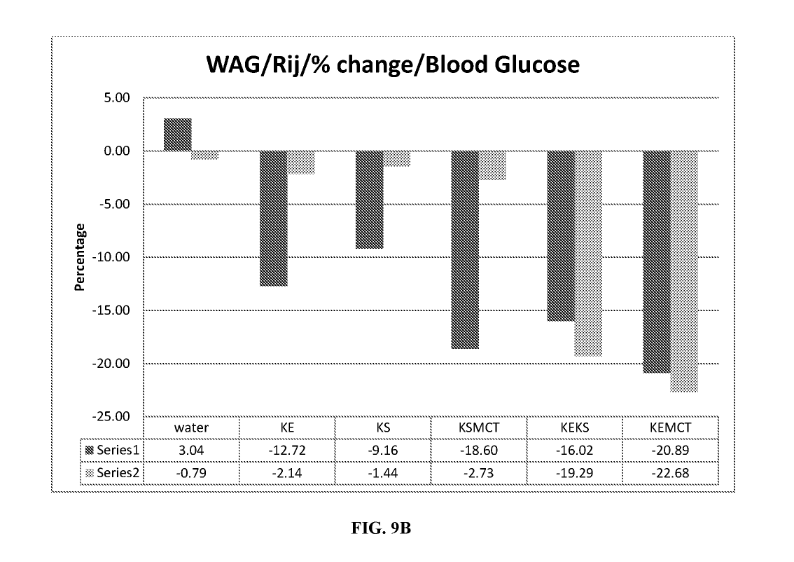

All dietary treatment groups exhibited a significant decline in blood glucose levels (FIG. 7A and FIG. 7B), as well as a significant increase in blood βHB levels (FIG. 78 and FIG. 8B) by 60 minutes after oral gavage, relative to baseline levels for each treatment group.

The results of this example demonstrate that WAG/Rij rats exhibit a decline in blood glucose levels and an increase in the blood levels of ketone bodies 60 minutes after administration of the ketogenic compounds.

### Example 6—Glucose Reduction Following Exercise in WAG/Rij Rats

The ability of the disclosed compositions to reduce blood glucose levels following exercise was subsequently tested in WAG/Rij rats. Rats were divided into KE, KS, KSMCT, KEKS, and KEMCT groups and gavaged with their respective ketogenic compounds described in Example 3. Animals were gavaged with their respective ketogenic compounds every day for 7 days.

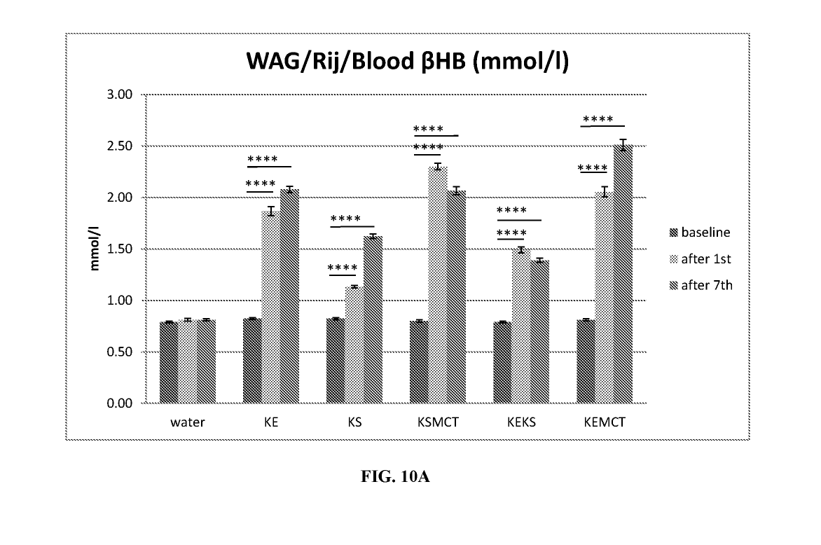

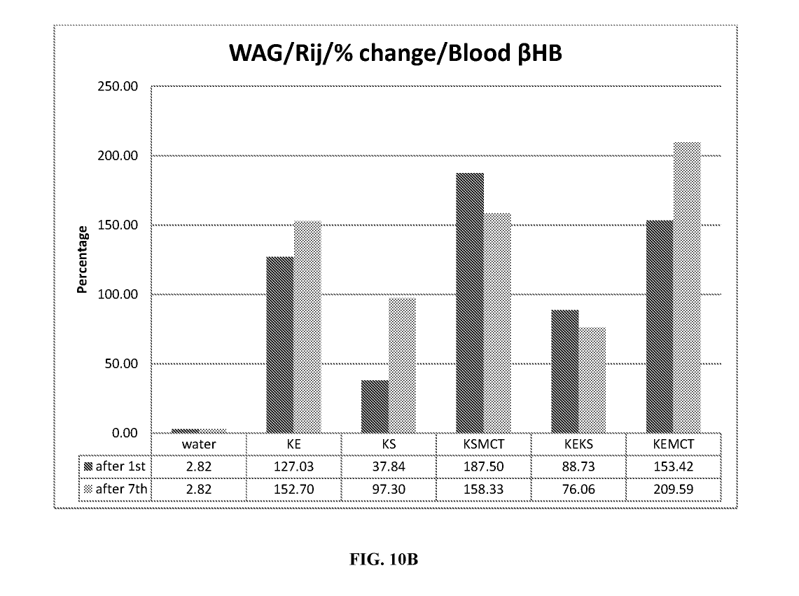

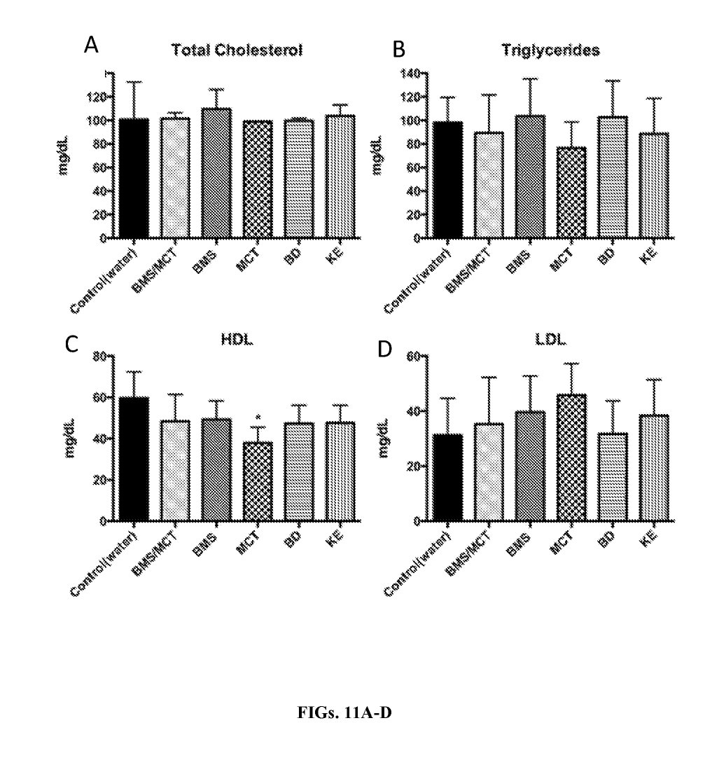

Following dietary treatment, animals were subjected to exercise on the accelerating rotarod. The rotarod was set to accelerate to 40 rpm in 180 seconds. The rats were exercised for 3 trials, with a 2 minute rest period between each trial. Following the third trial, blood samples were taken to evaluate blood levels of glucose and BHB (approximately 60 minutes after oral gavage administration). All groups exhibited a significant decline in blood glucose levels by 60 minutes after the first oral gavage treatment. At the day 7 time point, blood glucose levels remained below the baseline level only in KEKS and KEMCT groups (9A and 9B). Dietary treatment also caused a significant increase in blood levels of βHB following the treatment on day 1, and the treatment on day 7 in all treatment groups (FIGS. 10A and 10B).

### Example 7—Effect of Ketogenic Compounds on Triglycerides and Lipoproteins

The effect of the disclosed compositions on triglyceride and lipoprotein profile was assessed in Sprague-Dawley rats. Juvenile male Sprague-Dawley rats (275-325 g, Harlan Laboratories) were randomly assigned to one of six study groups: control (water, n=11), BD (n=11), KE (n=11), MCT (n=10), BMS (n=11), or BMS+MCT (n=12). On days 1-14, rats received a 5 g/kg body weight dose of their respective treatments via intragastric gavage. Dosage was increased to 10 g/kg body weight for the second half of the study (days 15-28) for all groups except BD and KE to prevent excessive hyperketonemia (ketoacidosis). Each daily dose of BMS would equal ˜1000-1500 mg of βHB, depending on the weight of the animal. Intragastric gavage was performed at the same time daily, and animals had ad libitum access to standard rodent chow 2018 (Harlan Teklad) for the duration of the study. The macronutrient ratio the standard rodent chow was 62.2, 23.8 and 14% of carbohydrates, protein and fat respectively. Baseline measurements showed no significant changes in triglycerides or the lipoproteins.

Triglyceride and lipoprotein concentrations measured after 4 weeks of daily exogenous ketogenic compound. No significant change in total cholesterol was observed at 4 weeks for any of the ketone treatment groups compared to control (FIG. 11A). No significant difference was detected in triglycerides for any ketogenic compound compared to control (FIG. 11B). MCT supplemented animals had a significant reduction in HDL blood levels compared to control (p<0.001) (FIG. 11C). LDL levels in ketone-supplemented animals did not significantly differ from controls (FIG. 11D).

### Example 8—Effect of Ketogenic Compounds on Blood Glucose in G1D Mice

GLUT1 deficiency syndrome (G1D) is characterized by impaired glucose transport in the brain and diagnosed with CSF glucose value of <2.2 mmol/L. There is no cure for G1D, but the ketogenic diet (KD) is often effective for the drug-resistant seizures and impairment of motor function associated with the disease. The effect of ketogenic compounds on blood glucose levels was assessed in glucose transporter type 1 (G1D)-deficient mice. A group of 48 G1D mice, aged 2-5 months old, were fed for 10 weeks with either a standard rodent diet (SD), a ketogenic diet (KD), a standard diet supplemented with 10% ketone ester (SD+KE) or a standard diet supplemented with 20% ketone mineral salt (SD+KS).

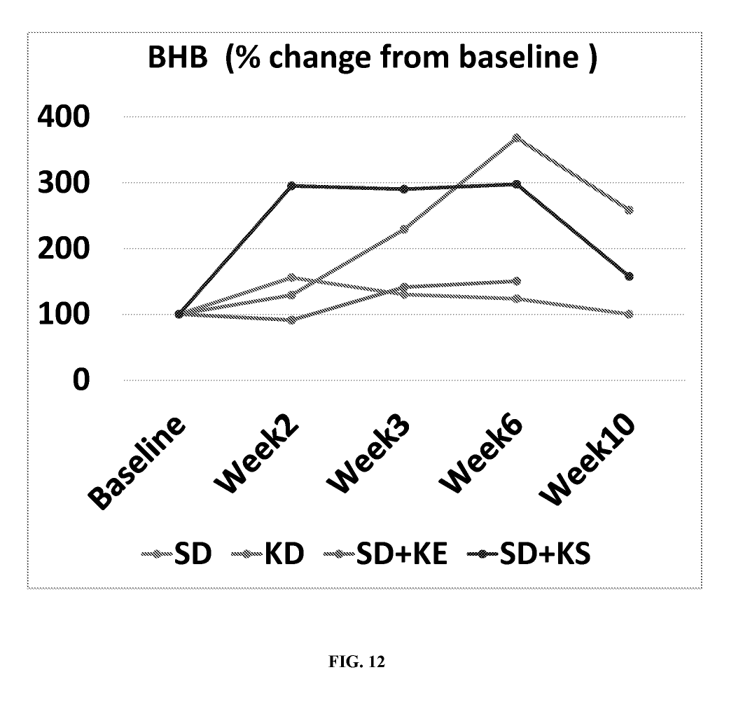

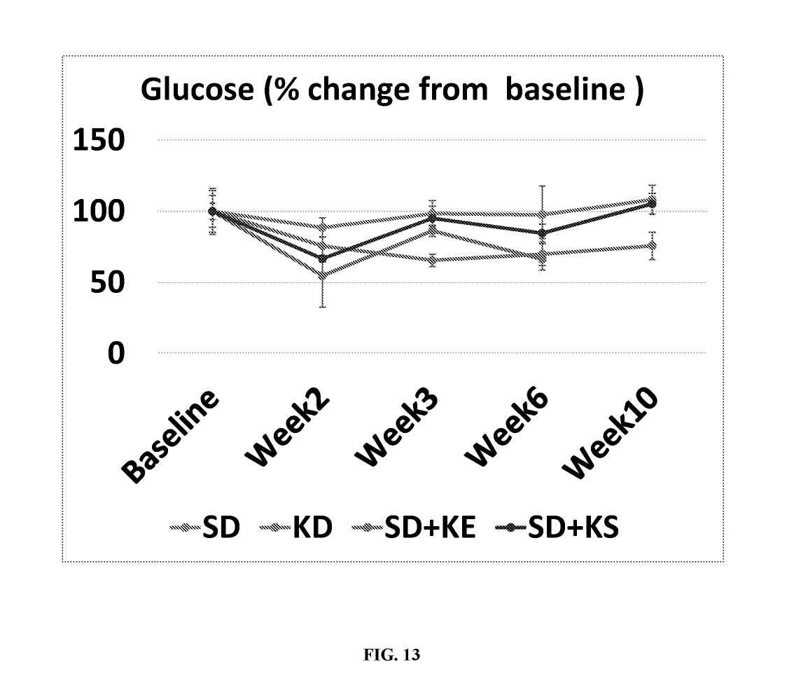

As shown in FIG. 12, KD caused slow increase, but high blood ketone levels. SD+KE caused slow and slight elevation, while SD+KS caused a significant and rapid elevation of blood ketone level from week 2, which level remained high until week 10 (FIG. 11). As shown in FIG. 13, glucose level quickly dropped and remained the lowest in SD+KE group. KD caused slow reduction (week 3, p=0.011) of blood glucose levels which remained low (week 10, p=0.05), while SD+KS caused an early decrease (week 2, p=0.04) in glucose levels.

For reasons of completeness, various aspects of the invention are set out in the following numbered clauses:

Clause 1: A method of reducing blood glucose levels in a subject, the method comprising administering a composition comprising one or more ketogenic compounds to the subject.

Clause 2: The method of clause 1, wherein the composition comprises one or more ketogenic compounds selected from the group consisting of a ketone ester, a ketone salt, a ketone body precursor, and a combination thereof.

Clause 3: The method of clause 2, wherein the one or more ketogenic compounds is a ketone ester.

Clause 4: The method of clause 3, wherein the ketone ester is 1,3-butanediol-acetoacetate diester.

Clause 5: The method of clause 3, wherein the composition further comprises a medium chain triglyceride.

Clause 6: The method of clause 5, wherein the ketone ester and the medium chain triglyceride have an approximate 1:1 ratio.

Clause 7: The method of clause 2, wherein the one or more ketogenic compounds is a ketone salt.

Clause 8: The method of clause 7, wherein the ketone salt is a β-hydroxybutyrate salt.

Clause 9: The method of clause 8, wherein the ketone salt is a β-hydroxybutyrate mineral salt.

Clause 10: The method of clause 9, wherein the β-hydroxybutyrate mineral salt is a Na⁺/K⁺ β-hydroxybutyrate mineral salt.

Clause 11: The method of clause 7, wherein the ketone salt comprises 50% of a solution.

Clause 12: The method of clause 7, wherein the ketone salt comprises a 50% solution of 375 mg/g of βHB per 125 mg/g of the ketone salt.

Clause 13: The method of clause 7, wherein the ketone salt is administered in a dose of about 1000 mg to about 1500 mg.

Clause 14: The method of clause 7, wherein the ketone salt further includes a medium chain triglyceride.

Clause 15: The method of clause 14, wherein the ketone salt and the medium chain triglyceride have an approximate 1:1 ratio.

Clause 16: The method of clause 2, wherein the composition comprises a ketone ester and a ketone salt.

Clause 17: The method of clause 16, wherein the ketone ester is 1,3-butanediol-acetoacetate diester and the ketone salt is a β-hydroxybutyrate salt.

Clause 18: The method of clause 1, wherein the composition is administered orally.

Clause 19: The method of any of clauses 1-18, wherein the composition reduces blood glucose levels in the subject without exercise.

Clause 20: The method of any of clauses 1-18, wherein the composition reduces blood glucose levels in the subject with exercise.

Clause 21: The method of clause 1, wherein the subject is a vertebrate.

Clause 22: The method of clause 21, wherein the mammal is a human.

1. A method of reducing blood glucose levels in a subject, the method comprising administering a composition comprising one or more ketogenic compounds to the subject.

2. The method of claim 1, wherein the composition comprises one or more ketogenic compounds selected from the group consisting of a ketone ester, a ketone salt, a ketone body precursor, and a combination thereof.

3. The method of claim 2, wherein the one or more ketogenic compounds is a ketone ester.

4. The method of claim 3, wherein the ketone ester is 1,3-butanediol-acetoacetate diester.

5. The method of claim 3, wherein the composition further comprises a medium chain triglyceride.

6. The method of claim 5, wherein the ketone ester and the medium chain triglyceride have an approximate 1:1 ratio.

7. The method of claim 2, wherein the one or more ketogenic compounds is a ketone salt.

8. The method of claim 7, wherein the ketone salt is a β-hydroxybutyrate salt.

9. The method of claim 8, wherein the ketone salt is a β-hydroxybutyrate mineral salt.

10. The method of claim 9, wherein the β-hydroxybutyrate mineral salt is a Na⁺/K⁺β-hydroxybutyrate mineral salt.

11. The method of claim 7, wherein the ketone salt comprises 50% of a solution.

12. The method of claim 7, wherein the ketone salt comprises a 50% solution of 375 mg/g of βHB per 125 mg/g of the ketone salt.

13. The method of claim 7, wherein the ketone salt is administered in a dose of about 1000 mg to about 1500 mg.

14. The method of claim 7, wherein the ketone salt further includes a medium chain triglyceride.

15. The method of claim 14, wherein the ketone salt and the medium chain triglyceride have an approximate 1:1 ratio.

16. The method of claim 2, wherein the composition comprises a ketone ester and a ketone salt.

17. The method of claim 16, wherein the ketone ester is 1,3-butanediol-acetoacetate diester and the ketone salt is a β-hydroxybutyrate salt.

18. The method of claim 1, wherein the composition is administered orally.

19. The method of claim 1, wherein the composition reduces blood glucose levels in the subject without exercise.

20. The method of claim 1, wherein the composition reduces blood glucose levels in the subject with exercise.

21. The method of claim 1, wherein the subject is a vertebrate.

22. The method of claim 21, wherein the vertebrate is a human.
